(19) United States
(12) Patent Application Publication
(10) Pub. No.: US 2013/0034893 A1
(43) Pub. Date: Feb. 7, 2013

(54) Methods for Coupling of Molecules to Metal/Metal Oxide Surfaces

(71) Applicants: Zhiyu Li, Woodstock, MD (US); Thomas Russell, Doylestown, PA (US); Jun Gao, Jinan (CN)

(72) Inventors: Zhiyu Li, Woodstock, MD (US); Thomas Russell, Doylestown, PA (US); Jun Gao, Jinan (CN)

(21) Appl. No.: 13/566,003

(22) Filed: Aug. 3, 2012

Related U.S. Application Data
(60) Provisional application No. 61/515,348, filed on Aug. 5, 2011.

(51) Int. Cl. C07K 1/107; C07K 17/02; C07K 14/00; C12N 11/14; C07K 14/765

(52) U.S. Cl. 435/176; 530/402; 530/363; 530/400; 530/391.3

(57) ABSTRACT

Functionalized magnetic particles are emerging as a reliable and convenient technique in the purification of biomacromolecules (proteins and nucleic acids). We disclose a novel coupling procedure that can be used to create stable ferromagnetic nickel particles coated with Protein A for the affinity purification of antibody. The protein purification procedure is gentle, scalable, automatable, efficient and economical. By modifying the functional groups of amino acids in the protein coating, nickel particles can be used not only for affinity purification but for other sample preparation and chromatographic applications as well including nucleic acid isolations. The method can be easily modified for small and medium scale antibody purification in lab and pre-clinical research.

### CROSS REFERENCE TO RELATED APPLICATIONS

Priority is claimed to U.S. Provisional Application Ser. No. 61/515,348 filed Aug. 5, 2011, the disclosure of which is incorporated by reference herein in its entirety.

### BACKGROUND OF THE INVENTION

1. Field of the Invention

This invention relates to improved procedures for binding molecules such as proteins to metal/metal oxide surfaces. More specifically the invention relates to methods for coupling proteins to nickel particles that are both magnetic and dense. These particles are used for separation procedures including protein purifications (affinity and ion exchange), isolation of nucleic acids, and enrichment or detection of chemicals

2. Description of Related Art

Immobilization (crosslinking) of proteins to solid supports has been practiced in biotechnology for applications in chromatography, biosensors, and diagnosis. Proteins can be immobilized to selected supports or matrixes by either covalent reaction or non-covalent binding. Based on the properties of supporting matrix and the purpose of applications, a proper immobilization method can be selected. Generally, the supporting matrixes can be classified into organic supports and inorganic supports. Organic supports include natural polymers (polysaccharides, proteins, and carbon) and synthetic polymers (polystyrene, polyacrylate, polyamide, vinyl, etc.). Inorganic supports include natural minerals (bentonite, silica) and processed materials (glass, metals, metal oxides). Non-covalent binding happens through non-specific interactions, such as electrostatic or hydrophobic interactions. These physical interactions are reversible depending on pH and ionic conditions. Therefore, protein immobilization through non-covalent interaction may not be suitable for harsh conditions that may cause the leaching of proteins from supporting matrixes.

A variety of reactions have been developed to crosslink protein to available functional groups on matrixes. The functional groups of proteins that can be used are carboxyl, amine, thiol, and hydroxyl groups. Proteins with posttranslational modification may possess more chemical groups for crosslinking, such as saccharides. The supporting matrixes also need to be modified to provide activated functional groups. The covalent crosslinking can be conducted by radial polymerization (e.g. poly(2-hydroxyethyl methacrylate (pHEMA)), aldehydes (e.g. glutaraldehyde), addition reactions (e.g. divinylsulfone), condensation reactions (e.g. carbodiimides (EDC)), high energy irradiation, and enzyme-mediated reactions (e.g. transglutaminase).

It is easy to utilize the functional groups of polymers for protein crosslinking However, there is no obvious chemical functional group available for inorganic metal particles (e.g. gold and nickel) and metal oxide (e.g. magnetite (Fe3O4; nickel oxide)). The common approaches to crosslink proteins or ligands to magnetite are to coat magnetite with polymers first and then immobilize proteins to the functional groups of coated polymers. There are several ways to compose magnetic polymer microsphere, 1) coating a single magnetic particle with polymers; 2) encapsulating multiple magnetic particles with polymers; 3) attaching multiple magnetic particles to the polymer particles. These techniques have been well developed. In addition, some approaches add chemicals with desired functional groups during the chemical synthesis of magnetite. Following this approach, it is possible to obtain precipitated magnetite with uniform size and make protein-coated magnetic particles at nano-scale. However, all of the above approaches require sophisticated chemical processing and are hard to scale up for industrial applications.

Protein-coated nickel or metal particles can be applied in a variety of chromatography applications, especially antibody affinity purification. Antibody therapy has become the dominant therapeutic class of biotherapeutic molecules and is used to treat many life threatening diseases [H. Samaranayake, T. Wirth, D. Schenkwein, J. K. Raty, S. Yla-Herttuala, Ann Med 41 (2009) 322]. However, the production of therapeutic antibodies is quite costly both in terms of capital and variable costs. The downstream processing in antibody purification contributes significantly to the high cost of antibody therapy [D. Low, R. O'Leary, N. S. Pujar, J Chromatogr B Analyt Technol Biomed Life Sci 848 (2007) 48; S.S. Farid, J Chromatogr B Analyt Technol Biomed Life Sci 848 (2007) 8; U. Gottschalk, Biotechnol Prog 24 (2008) 496]. Among the technologies evaluated to tackle this problem, magnetic adsorbent-based bioseparation is showing promise but has not yet proven economically viable. Magnetic-based protein separation is becoming the routine for biological research, immunoassays, and diagnostics. Magnetic absorbent particles are usually made by encapsulating nano to micro-meter magnetic particles (iron oxide superparamagnetic particles) inside polymers (polystyrene, methylmethacrylate, silica, etc.) followed by covalent modification (Protein A and ligand) on the polymer surface) [A. A. Neurauter, M. Bonyhadi, E. Lien, L. Nokleby, E. Ruud, S. Camacho, T. Aarvak, Adv Biochem Eng Biotechnol 106 (2007) 41; O. Olsvik, T. Popovic, E. Skjerve, K. S. Cudjoe, E. Homes, J. Ugelstad, M. Uhlen, Clin Microbiol Rev 7 (1994) 43; R. X. Rui Hao, Zhichuan Xu, Yanglong Hou, Song Gao, and Shouheng Sun, Advanced materials 22 (2010) 2729]. Using magnetic sorbents in antibody affinity purification may reduce operational time, and eliminate steps in protein/cell harvest [M. Franzreb, M. Siemann-Herzberg, T. J. Hobley, O. R. Thomas, Appl Microbiol Biotechnol 70 (2006) 505]. Pilot research has shown the advantages of magnetic separation over conventional chromatography in preparative purification of antibodies [K. Holschuh, A. Schwammle, Journal of Magnetism and Magnetic Materials (2005) 345; J. J. Hubbuch, D. B. Matthiesen, T. J. Hobley, O. R. Thomas, Bioseparation 10 (2001) 99]. Although many methods have been applied to the production of magnetic sorbents, the poor physical characteristics of the first generation of magnetic beads coupled with their high manufacturing cost have limited the industrial application of this approach.

It has been shown in the art that proteins are able to adhere directly to nickel particle surfaces and form relatively stable complexes without covalent binding of protein to nickel [D. Liu, Y. wang, W. Chen, Colloids and Surfaces B: Biointerfaces 5 (1995) 25; W.-Y. C. Hwai-Shen Liu Colloids and Surfaces B: Biointerfaces 5 (1995) 35; K. Swinnen, A. Krul, I. Van Goidsenhoven, N. Van Tichelt, A. Roosen, K. Van Houdt, J Chromatogr B Analyt Technol Biomed Life Sci 848 (2007) 97]. The mechanism by which these complexes form is not fully understood. Leaching of coated proteins (i.e., Protein A) during elution of target proteins from nickel particles (using high or low pH buffer) would contaminate the isolated target proteins, and leaching during any of the process steps would slowly decrease the capacity of the particles over time and thereby their half-life. The disclosed method is able to minimize protein leaching during antibody capturing, washing, and elution. The antibody binding affinity of the coated and immobilized Protein A is not affected. Moreover, protein-coated nickel particles can be kept at 4° C. for at least 2 years without losing antibody binding functions.

Although coating nickel particles with proteins or peptides is a quick and efficient process, we needed to determine whether the complexes were sufficiently stable to withstand elution conditions to release the antibodies from the Protein A. We disclose a method of crosslinking the nickel-bound protein ligands to prevent leaching during elution of target proteins. The following invention disclosed herein details methods that overcome the limitations of superparamagnetic particles of the art and naked nickel particles of the art.

### SUMMARY OF THE INVENTION

A novel magnetic particle (U.S. patent application Ser. No. 11/159,957, incorporated herein by reference) was used to develop the coupling procedure disclosed herein. These solid metal particles such as nickel particles, but not limited to, are very dense (˜9 g/cm³) and strongly magnetic and can be heated to temperatures that lead to nickel particles with a metal oxide coating. The particles have irregular surfaces which increase the surface area and potentially the binding capacity. In addition, the particles can be manufactured in a broad range of sizes for different applications. The significantly higher density than other magnetic particles enhances mixing and thereby product capture in undiluted, viscous solutions. Ligand-functionalized nickel particles have rapid binding kinetics to target proteins, and, because they are strongly magnetic, their separation times are also very rapid. This separation technology can be performed in small lab scale volumes or scaled up to volumes necessary for manufacturing-scale protein purification. The particles' physical properties, density, rapid binding and strong magnetic moment, minimize overall sample exposure time and reduce the potential for nonspecific protein alteration or degradation during downstream processing.

Without stable matrixes, it is impossible to covalently immobilize proteins to metal particles. However, proteins can form non-covalent interaction with metallic particles through non-specific electrostatic or hydrophobic interactions. These non-covalent coated proteins can then be crosslinked with techniques described above to form sable matrixes as a net evenly covering a particle. These protein matrixes possess functional groups, such as amine, carboxyl, thiol, hydroxyl, and aldehyde. It provides a variety of choices for protein or ligand crosslinking to proteins matrixes coated to particles. Following this approach, it is possible to covalently modify metal particles (indirectly through stabilized protein matrixes) and other particles or material (carbon nanotube) that lack of chemical functional groups for modifications without polymer coating and chemical synthesis. In addition to albumin, proper proteins, peptides, and ligands can be screened for strong non-covalent binding and then be crosslinked and utilized as stable matrixes. These proteins can be cysteine-rich protein, basic protein, acid protein, hydrophobic protein, or combination of selected properties (e.g. genetically engineered proteins and synthetic peptides with all these properties) for the strong binding and easy crosslinking

In one embodiment of the invention incorporates affinity purification of proteins or cation/anion exchange chromatography. We have found that more active, stable nickel particles bound with desired molecules will allow the capture of a desired protein such as, but not limited to, monoclonal antibody by affinity purification, a nucleic acid of interest, or targeted chemicals, all of which can be created by following standard crosslinking procedures found in the art. The improvement being the inclusion of protein already bound to the particle or an indifferent protein i.e. bovine serum albumin incorporated into the crosslinking reaction. Basic proteins, such as histone proteins and protamine, can be incubated and crosslinked directly and used for cation exchange chromatography. Acid proteins, such as trypsin inhibitor or pepsin A, can be incubated and crosslinked directly and used for anion exchange chromatography.

In another embodiment of the invention the protein i.e. albumin, but not limited to, bound particles can be heated to high temperatures that permit stable protein metal/metal oxide surface bonds through covalent bonds between the metal oxides on the surface and active groups on the protein to enhance the interactions between proteins and the metallic surface. Therefore, crosslinking would not be required to form a stable matrix for chemical modification. The reactive groups of the bound protein can then be used to covalently bind other molecules of interest i.e. proteins, nucleic acids, or chemicals to the particles.

### BRIEF DESCRIPTION OF DRAWINGS

FIG. 1. Leaching of BSA from nickel particles in acid and alkaline elution. Leached BSA was visualized by SDS-PAGE and silver staining

FIG. 2. Protein A-coated nickel particles are able to capture antibody. Leached Protein A and eluted antibody were visualized by SDS-PAGE followed by silver stain.

FIG. 3. Flowchart of affinity purification using crosslinked protein-bound Nickel particles.

FIG. 4. Crosslinking of Nickel-bound BSA and Protein A can minimize protein leaching from particles during acid or alkaline elution.

FIG. 5. Cross-linked Protein A bound to nickel particles can be used in IgG affinity purification with barely detectable Protein A leaching (SDS-PAGE and silver stain).

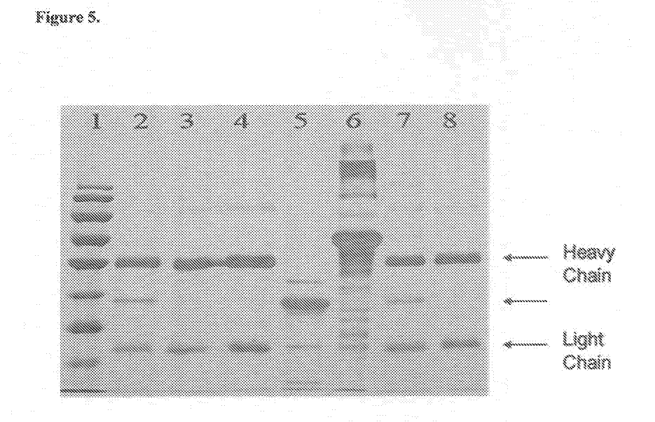

FIG. 6. Protein A-coated nickel particles can quickly isolate antibody from serum (SDS-PAGE and silver stain).

### DETAILED DESCRIPTION OF PREFERRED EMBODIMENTS

Nickel particles (˜3 μm) were provided by Russell Biotech Inc. BSA and glutaraldehyde was purchased from Sigma Aldrich. Protein A was purchased from Biovison. Purified mouse IgG and mouse serum were purchased from Jackson ImmunoResearch. Bradford protein assay reagent was purchased from BioRad. BCA protein assay kit was purchased from PIERCE.

### Preparation of Protein-Coated Nickel Particles

Nickel particles (1 g), bed volume between 0.4-0.5 ml, were washed 3 times for 5 minutes each with 3 ml PBS. The equilibrated particles were then incubated with 2 ml of 0.1% BSA or Protein A (1mg/ml) at 4° C. overnight. For Protein A coating, the Protein A solution was removed after overnight incubation and followed by blocking with 2 ml 0.1% BSA for 2 hours at room temperature. Protein-coated nickel particles were then washed 3 times for 5 minutes each with 3 ml PBS and stored at 4° C. until use.

### Crosslinking of Protein-Coated Nickel Particles

After overnight incubation with BSA or Protein A, 1% glutaraldehyde was added to protein/nickel mixtures at a 1:60 molar ratios between proteins and glutaraldehyde. The mixtures were shaken at 250 RPM for 2 hours at 37° C. The reaction was terminated by adding 1/10 volume of 1 M Tris-HCl (pH 8.0), and the particles were washed 3 times with 3 ml PBS.

### IgG Binding and Elution

Protein A-coated nickels particles (1 g) were incubated with 0.1% mouse IgG or 1 mg IgG/ml mouse serum for 5, 10, 20, 30, 40 and 50 minutes at room temperature. After incubation, the nickel particles were magnetically removed from the solution. Nickel particles were degaussed and washed 3 times for 5 minutes each with 1X PBS. Protein A-bound IgG was eluted by adding 500 pl acid buffer (100 mM citric acid, pH 2.2) or alkaline buffer (100 mM triethanolamine, pH 12.8) and rotated at 20 rpm for 5 minutes at room temperature. After elution, particles were magnetically removed from solution, and supernatants were neutralized by adding 75 μl A of 1M Tris-HCl (pH 8.0). Concentration of eluted IgG was obtained by Bradford and BCA methods. Proteins were visualized by SDS-PAGE followed by silver or Coomassie Blue G-250 stain.

Leaching of BSA Adsorbed onto Nickel Particles

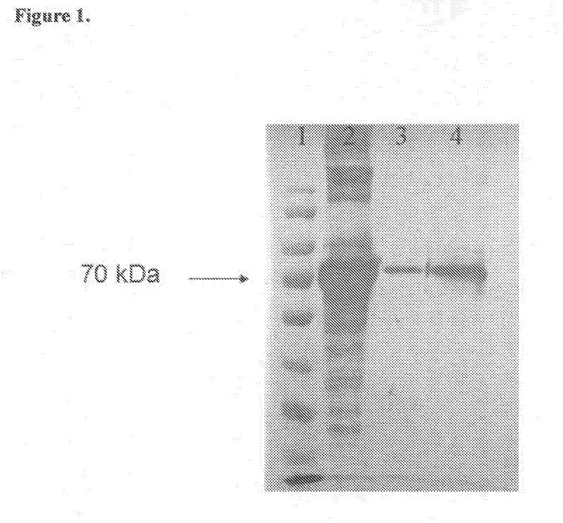

Although the mechanism is unclear, proteins adhere to the nickel particles and form relatively stable complexes without being covalently attached to the nickel surface. Since leaching of proteins from nickel would limit chromatographic utility, we tested the amount of protein released from the particles under different incubation conditions. BSA was used as a model protein to evaluate the leaching (FIG. 1). BSA-bound nickel particles were incubated with acid buffer (lane 2, 100 mM citric acid, pH 2.2) and alkaline buffer (lane 3, 100 mM triethylamine, pH 12.8), respectively. Supernatants were collected, and eluted BSA was identified by SDS-PAGE and silver staining BSA bound to the nickel particles was calculated, and BSA leached into elution buffers was measured. These measurements indicated that less than 2% of nickel-bound BSA eluted in acid buffer, and about 2-5% eluted in alkaline buffer.

Leaching of Protein A During Antibody Purification

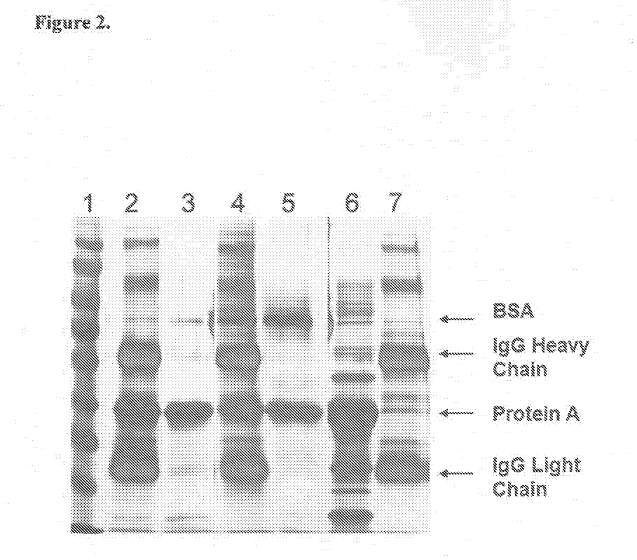

Protein A, like BSA, forms non-covalent complexes with the surfaces of nickel particles. Since nickel-bound Protein A particles would be useful for magnetic purification of antibodies if leaching were minimal, we tested Protein A leaching under different conditions. Protein A nickel particles were incubated with 1 mg/ml of mouse IgG for 20 minutes. Particles containing the Protein A-IgG complexes were magnetically removed from the solution. Data in FIG. 2 demonstrate that Protein A-bound nickel particles were able to capture purified IgG. However, during IgG elution (lane 2 and 4) significant amounts of

Protein A leached from the particles. Furthermore, Protein A eluted from the nickel surface more readily than BSA in both acid and alkaline conditions (lane 3 and 5).

### Crosslinking of Nickel-Bound Proteins

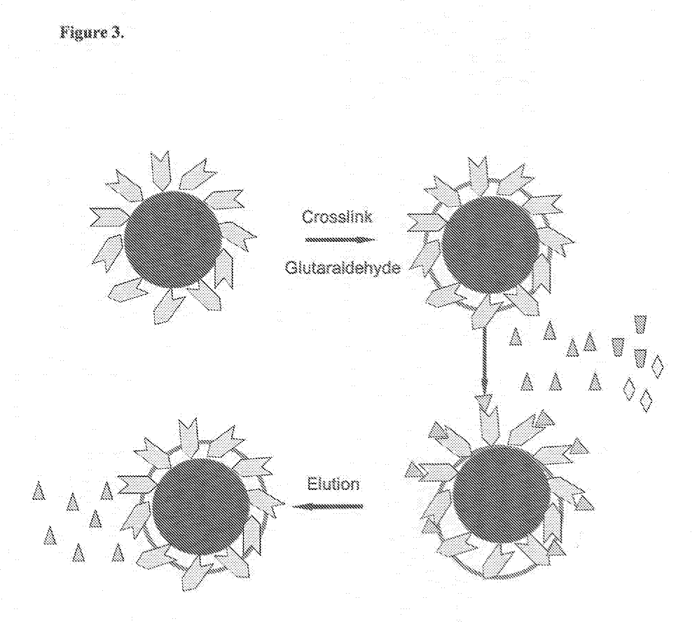

Because both Protein A and BSA were found to leach from the nickel particles during elution of the target protein, a more stable complex of these proteins to the nickel was required. Since there is no obvious functional group on nickel surface, it is not practical to covalently conjugate Protein A to nickel particles as might be done with polymer-coated magnetic particles currently the art. We tested whether crosslinking nickel-bound proteins could stabilize protein binding and prevent or, at least, minimize leaching (FIG. 3). If crosslinking nickel-bound proteins/polymers could be demonstrated to provide stable matrices for further chemical modification on chemical functional groups presented by proteins/polymers, it would dramatically expand the application of nickel particles in, protein purification, and ligand/chemical detection.

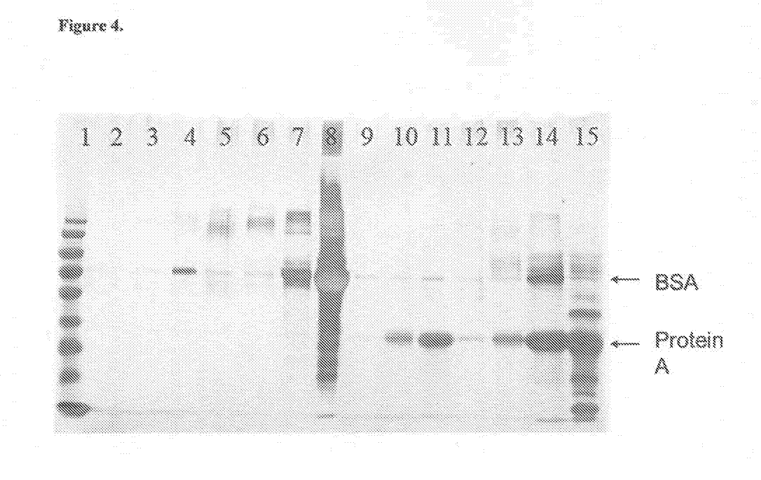

Glutaraldehyde was used to crosslink nickel-bound BSA and Protein A to the nickel particle surface (FIG. 4). Either BSA (lanes 2-7) or Protein A (lanes 9-14) were bound to the nickel particles. Protein A particles were then blocked for 2 hr with BSA. The glutaraldehyde crosslinking was performed in the presence of 0.1% BSA (lane 2 and 5) or Protein A (lanes 9 and 12) or in PBS alone (lane 3, 6, 10, and 13). The crosslinking in the presence of proteins in solution showed the least leaching in elution buffer (lane 2, 5, 9, and 12) not currently done in the art. Alkaline elution (lanes 6, 7, 13 and 14) caused more leaching than acid elution (lanes 2, 3, 9 and 10). As before, under the same conditions, leaching of BSA from the nickel particles was much less than that of Protein A.

### Heating Following Absorption to Metal Surface

Another aspect of the invention is that Nickel particles bound with BSA or other proteins can be heated to temperatures not possible with today's magnetic separation technology, thus yielding stable (especially after netting as described above) denatured polypeptides and chemical groups (NH2, COOH, SH etc.) that can be used for covalent attachment of proteins, nucleic acids, or chemicals directly. The functionalized nickel particles can be utilized in all kinds of chromatographic separations.

### EXAMPLES

1. Antibody Isolation from Mouse Serum

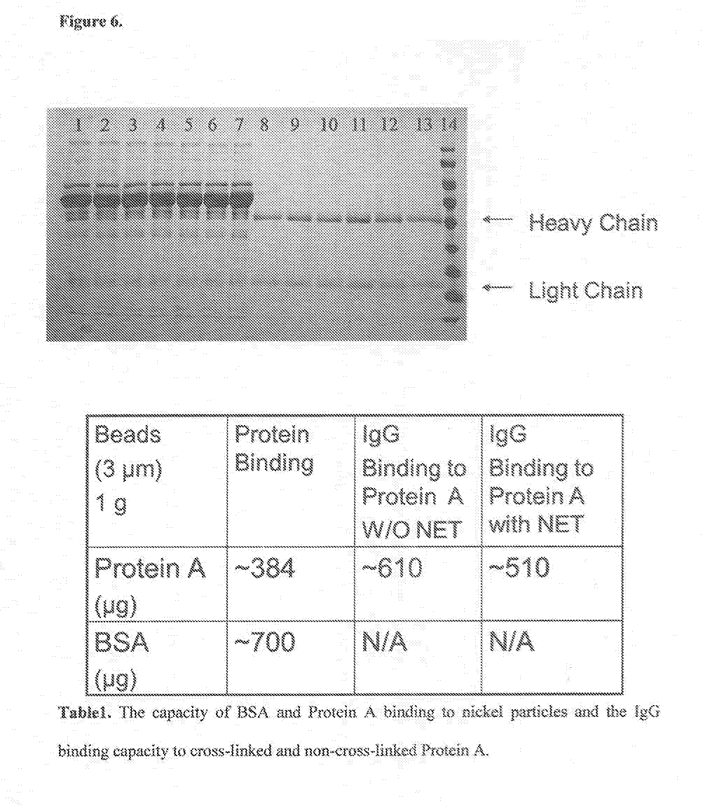

Protein A-bound nickel particles have very rapid mixing and capture kinetics. Therefore, they can also be used to isolate antibody from undiluted mouse serum more rapidly than other magnetic beads. Mouse serum was incubated with crosslinked Protein A-bound nickel particles for the times indicated in FIG. 6. In as few as 5 minutes, the Protein A-bound nickel particles efficiently isolated antibodies from the serum. Very little additional antibody was captured by incubation times up to 50 minutes. The purity and yield of antibody were not compromised because of the short incubation time. In addition, the magnetic removal of particle antibody complexes from the serum occurs in less than 1 minute. This process is significantly more rapid than that of other Protein A-based modified chromatographic substrates, such as agarose beads. This increased rate of reaction may be due to the non-porous nickel surface, or it may be due the rapid mixing of the dense particles in the viscous solution.

Excellent purity, simple procedure and high yield are attractive characteristics for scaling up antibody purification from crude serum or cell extracts. In addition, short incubation times may significantly decrease contamination during antibody purification.

### 2. Coating Carbon Nano-Tubes

The disclosed coupling procedure will be used to coat carbon nano-tubes with desired molecules. Carbon nano-tubes of various sizes and shapes will be non-covalently bound with the desired molecules by absorption in an appropriate buffer solution. The carbon nano-tubes will then be washed by centrifugation and resuspended in buffer. The crosslinking agent, glutaraldeyhyde or another appropriate crosslinking agent will be added to the solution in the presence of the molecule bound to the carbon nano-tube by absorption. The reaction will be carried out at room temperature for various times. The reaction will be stopped by the addition of glycine as in the art. The modification of functional groups of nano-tubes—coated proteins with molecules of interest, such as proteins (antibodies and ligands), nucleic acids (DNA, RNA, or chemically modified DNA or RNA derivatives), or chemicals (drugs or chemical probes) will be studied.

### 3. Formation of the Metal Oxide

Nickel particles will be heated to 250 deg centigrade for 3-72 hours to form a metal oxide layer. The particles will be cooled to room temperature (RT). Various BSA solutions ranging from 0.1% to 2% in 50 mM Tris buffer pH 8.0 will be mixed with 32-64 mg/ml nickel particles and mixed end-over-end over night at RT. The particles will be rinsed in Tris buffer 3 times and heated from 56 degree C. and higher to determine the optimal temperature to form a stable covalently bound BSA- nickel particle. The parameter to be measured to determine stability will be an acid and base elution to determine the temperature at which no BSA is eluted by acid or base. These BSA-nickel particles will then be used to covalently couple molecules of interest i.e. but not limited to, antibodies to reactive side chains on BSA by standard procedures know in the art.

Although the present invention has been described with reference to specific embodiments, workers skilled in the art will recognize that many variations can be made therefrom. It is to be understood and appreciated that this discovery in accordance with this invention are only those which are illustrated of the many additional potential variations that can be envisioned by one of ordinary skill in the art, and thus are not in any way intended to be limiting of the invention. Accordingly, other objects and advantages of the invention will be apparent to those skilled in the art from the description together with the claims.

What is claimed is:

1. A method for crosslinking molecules to metal particles comprising:
a. absorbing a molecule to the surface of a metal particle to form a non-covalently bound complex; and
b. adding a crosslinking reagent containing a stabilizing protein.

2. The method of claim 1 where the molecule is from a group consisting of a peptide, a protein, nucleic acid, and chemicals.

3. The method of claim 1 where the metal particle is nickel.

4. The method of claim 1 where the stabilizing protein is the same as the absorbing molecule.

5. The method of claim 1 where the stabilizing protein is bovine serum albumin.

6. The method of claim 1 further comprising washing of the complex to remove unbound molecules.

7. The method of claim 1 where the molecule is a basic protein for use in cation chromatography.

8. The method of claim 7 where the basic proteins are histone proteins or protamine.

9. The method of claim 1 where the molecule is an acid protein for use in anion chromatography.

10. The method of claim 9 where the acid protein is trypsin inhibitor or pepsin A.

11. A method for crosslinking molecules to metal particles comprising:
a. absorbing a molecule to the surface of a metal particle bound to a protein to form a non-covalently bound complex; and
b. adding a crosslinking reagent in amounts sufficient to stabilize the metal particles.

12. The method of claim 9 where the molecule is from a group consisting of a peptide, a protein, nucleic acid, and chemicals.

13. The method of claim 9 where the metal particle is nickel.

14. The method of claim 9 further comprising washing of the complex to remove unbound molecules.

15. A method for covalently coupling a protein to a metal/metal oxide surface of a particle comprising:
a. adsorbing a protein to the metal/metal oxide surface of a particle to form a non-covalent protein/particle complex;
b. removing unbound protein; and
c. heating the complex to a sufficient temperature to form covalent bonds between the adsorbed protein and oxides on the particle surface.

16. The method of claim 15 where the protein is bovine serum albumin.

17. The method of claim 15 where the metal particle is nickel.

18. The method of claim 15 wherein the covalently bound protein is further coupled to a second molecule by standard covalent coupling procedures.

19. The method of claim 18 wherein the second molecule is from a group consisting of proteins, nucleic acids and chemicals.

20. The method of claim 15 where the protein is a monoclonal or polyclonal antibody.
